(19) United States
(12) Patent Application Publication
(10) Pub. No.: US 2018/0078222 A1
(43) Pub. Date: Mar. 22, 2018

(54) SYSTEM HAVING A MOBILE CONTROL DEVICE AND METHOD FOR OUTPUTTING A CONTROL SIGNAL TO A COMPONENT OF A MEDICAL IMAGING APPARATUS

(71) Applicant: Siemens Healthcare GmbH, Ertangen (DE)

(72) Inventors: Thomas BOETTGER, Erlangen (DE); Thilo HANNEMANN, Erlangen (DE); Gerhard KRAEMER, Igensdorf (DE); Daniel LERCH, Weilersbach (DE); Carsten THIERFELDER, Pinzberg (DE); Fernando VEGA, Erlangen (DE); Selma ZEHNER, Forchheim (DE)

(73) Assignee: Siemens Healthcare GmbH, Erlangen (DE)

(21) Appl. No.: 15/695,305

(22) Filed: Sep. 5, 2017

(30) Foreign Application Priority Data
Sep. 21, 2016 (DE) 102016218138.6

(51) Int. Cl. A61B 6/04; G05B 19/048; G05B 19/402; G06F 19/00

(52) U.S. Cl. CPC A61B 6/04; G05B 19/048; G06F 3/167; G06F 19/3406; G05B 19/402

(57) ABSTRACT

An embodiment of the invention relates to a system. In an embodiment, the system includes a component of a medical imaging apparatus; a mobile control device including an operating element designed as an electromechanical switching element; and a control unit designed to output a control signal to the component. When the system is in an operating state, the mobile control device and the control unit are coupled such that an actuation of the operating element causes the control signal to be output to the component via the control unit.

### PRIORITY STATEMENT

The present application hereby claims priority under 35 U.S.C. § 119 to German patent application number DE 102016218138.6 filed Sep. 21, 2016, the entire contents of which are hereby incorporated herein by reference.

### FIELD

At least one embodiment of the invention generally relates to a system, comprising a component of a medical imaging apparatus and a mobile control device. At least one embodiment of the invention furthermore generally relates to a method for outputting a control signal to a component of a medical imaging apparatus. At least one embodiment of the invention furthermore generally relates to a use of a system for outputting a control signal to a component of a medical imaging apparatus.

### BACKGROUND

Medical imaging apparatuses, in particular sectional imaging medical devices such as for example CT devices and MR devices, typically require as precise positioning as possible of the transfer plate, on which a patient is supported, relative to the gantry. In many cases the positioning is carried out by using buttons or other control elements which are fixedly arranged directly on the gantry. Depending on the device, the buttons or other control elements are located exclusively on the front of the device or both on the front and also on the rear of the device.

The control elements are often arranged distributed in redundant fashion at a plurality of locations on the gantry, which consequently has a negative effect in respect of costs and complexity. Depending on the needs of the patient and cultural circumstances the limitation of only being able to perform the positioning and/or removal of the patient when standing directly beside the patient may constitute a severe restriction. It would thus be desirable for example in order to save time or also for reasons of patient safety, in particular in the event of an emergency, to be able to move the transfer plate as soon as the patient and/or the user enters the examination room. In the case of cultural or health restrictions it may likewise be expedient to control the patient positioning device from a position which is further away from the medical imaging apparatus.

### SUMMARY

At least one embodiment of the invention provides an alternative capability for controlling a component of a medical imaging apparatus.

Further advantageous embodiments of the invention are set down in the claims.

At least one embodiment of the invention relates to a system comprising

a component of a medical imaging apparatus,

a mobile control device having an operating element which is designed as an electromechanical switching element, and

a control unit which is designed in order to output a control signal to the component,

wherein when the system is in an operating state the mobile control device and the control unit are coupled in such a manner that an actuation of the operating element causes the control signal to be output to the component via the control unit.

In particular, a component of a system according to one of the embodiments which are disclosed in this description and/or in the claims, which is designed in order to perform a given step of a method according to one of the embodiments that are disclosed in this description and/or in the claims, can be implemented in the form of a hardware unit which is configured in order to perform the given step and/or which is configured in order to execute a computer-readable instruction in such a manner that the hardware unit can be configured via the computer-readable instruction in order to perform the given step. In particular, the system can comprise a storage area, for example in the form of a computer-readable medium in which computer-readable instructions are stored, for example in the form of a computer program.

### BRIEF DESCRIPTION OF THE DRAWINGS

Selected embodiments will be explained in the following with reference to the attached figures. The illustrations in the figures are schematic, highly simplified and not necessarily to scale.

In the drawings:

FIG. 1 shows a mobile control device of a system in accordance with a first embodiment of the invention,

FIG. 2 shows a first view of a mobile control device of a system in accordance with a second embodiment of the invention,

FIG. 3 shows a second view of a mobile control device of a system in accordance with the second embodiment of the invention,

FIG. 4 shows a mobile control device of a system in accordance with a third embodiment of the invention,

FIG. 5 shows a system in accordance with a fourth embodiment of the invention,

FIG. 6 shows a system in accordance with a fifth embodiment of the invention,

FIG. 7 shows a system in accordance with a sixth embodiment of the invention,

FIG. 8 shows a system in accordance with a seventh embodiment of the invention,

FIG. 9 shows a flowchart for a method for outputting a control signal to a component of a medical imaging apparatus in accordance with an eighth embodiment of the invention,

FIG. 10 shows a flowchart for a method for outputting a control signal to a component of a medical imaging apparatus in accordance with a ninth embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a system comprising

a component of a medical imaging apparatus,

a mobile control device having an operating element which is designed as an electromechanical switching element, and

a control unit which is designed in order to output a control signal to the component,

wherein when the system is in an operating state the mobile control device and the control unit are coupled in such a manner that an actuation of the operating element causes the control signal to be output to the component via the control unit.

The mobile control device can in particular be designed in such a manner that the mobile control device can be carried in one hand or on the body by a user and/or that the mobile control device can be operated by a user while the mobile control device is being carried in one hand and/or on the body by the user.

The mobile control device can in particular comprise a first audio interface which is designed in order to receive acoustic signals from the user and/or to output acoustic signals to the user.

The mobile control device can in particular comprise a plurality of touch-sensitive operating elements which together form a touch-sensitive panel that is designed in order to display at least one software operating element and/or to capture at least one touch gesture for actuating at least one software operating element.

The mobile control device can in particular comprise a display unit which is designed in order to display to the user data relating to the medical imaging apparatus and/or the component of the medical imaging apparatus.

The system can in particular furthermore comprise the following components:

at least one context determination unit which is designed in order to determine a context that relates to the medical imaging apparatus and/or a medical examination which can be carried out and/or is at least partially carried out via the medical imaging apparatus, and/or

a pose determination unit which is designed in order to determine a pose of the mobile control device.

The pose determination unit can in particular be designed in order to determine whether the pose of the mobile control device crosses a pose region boundary.

The system can in particular comprise an alarm signal output unit which is designed in order to output an alarm signal in such a manner that the alarm signal is output when the pose of the mobile control device crosses a pose region boundary.

The system can in particular furthermore comprise a configuration unit which is designed in order to configure the mobile control device and/or the control unit on the basis of the context and/or on the basis of the pose of the mobile control device.

The component of the medical imaging apparatus can in particular comprise an examination table and a transfer plate which is arranged on the examination table and can be moved relative to the examination table.

The mobile control device can in particular comprise a first transfer operating element,

wherein the mobile control device and/or the control unit can be configured via the configuration unit in such a manner on the basis of the pose of the mobile control device

that for at least a first pose of the mobile control device an actuation of the first transfer operating element causes the transfer plate to move in a first horizontal direction and that for at least a second pose of the mobile control device an actuation of the first transfer operating element causes the transfer plate to move in a second horizontal direction.

The pose determination unit can in particular be designed in order to determine whether the pose of the mobile control device is situated in a first pose region,

wherein the mobile control device and/or the control unit can be configured via the configuration unit in such a manner on the basis of the pose of the mobile control device

that an actuation of the operating element causes the control signal to be output to the component via the control unit depending on whether the pose is situated in the first pose region.

The pose determination unit can in particular be designed in order to determine whether the pose of the mobile control device is situated in a second pose region,

wherein the mobile control device and/or the control unit can be configured via the configuration unit in such a manner on the basis of the pose of the mobile control device

that the mobile control device and the control unit are coupled depending on whether the pose is situated in the second pose region.

The system can in particular comprise a tablet computer having a software application which is designed in order to control the medical imaging apparatus and/or the component of the medical imaging apparatus,

wherein the mobile control device comprises the tablet computer and/or wherein the mobile control device can be connected and/or is connected to the tablet computer via a mechanical connection and/or via a data transfer connection.

The system can in particular comprise a smartphone having a software application which is designed in order to control the medical imaging apparatus and/or the component of the medical imaging apparatus,

wherein the mobile control device comprises the smartphone and/or wherein the mobile control device can be connected and/or is connected to the smartphone via a mechanical connection and/or via a data transfer connection.

The system can in particular comprise the medical imaging apparatus, wherein the medical imaging apparatus comprises a docking unit which is designed in order to dock the mobile control device in releasable fashion.

In particular, the mechanical connection and/or the docking unit can comprise a magnetic holder.

At least one embodiment of the invention furthermore relates to a method for outputting a control signal to a component of a medical imaging apparatus, the method comprising:

coupling of a mobile control device, which comprises an operating element that is designed as an electromechanical switching element, and a control unit, which is designed in order to output a control signal to the component, in such a manner that an actuation of the operating element causes the control signal to be output to the component via the control unit,

actuation of the operating element, and

output of the control signal.

The method can in particular furthermore involve the following steps:

determination of a context which relates to the medical imaging apparatus and/or a medical examination that can be carried out and/or is at least partially carried out via the medical imaging apparatus, and/or determination of a pose of the mobile control device,

configuration of the mobile control device and/or of the control unit on the basis of the context and/or on the basis of the pose of the mobile control device.

The invention furthermore relates to the use of a system according to one of the embodiments which are disclosed in this description and/or in the claims in order to output a control signal to a component of a medical imaging apparatus, wherein the output of the control signal to the component via the control unit is effected by actuating the operating element.

The medical imaging apparatus can for example be chosen from the imaging modalities group including an X-ray device, a C-arm X-ray device, a computed tomography device (CT device), a molecular imaging device (MI device), a single-photon emission computed tomography device (SPECT device), a positron emission tomography device (PET device), a magnetic resonance tomography device (MR device) and combinations thereof (in particular PET-CT device, PET-MR device). The medical imaging apparatus can furthermore comprise a combination of an imaging modality, which is chosen for example from the imaging modalities group, and a radiation modality. In this situation the radiation modality can for example comprise an irradiation unit for therapeutic irradiation. Without limiting the generality of the invention, a computed tomography device is cited by way of example for a medical imaging apparatus in some of the embodiments.

According to an embodiment of the invention, the medical imaging apparatus comprises an acquisition unit which is designed in order to acquire the acquisition data. In particular, the acquisition unit can comprise a radiation source and a radiation detector. An embodiment of the invention provides that the radiation source is designed in order to emit and/or excite a radiation, in particular an electromagnetic radiation, and/or that the radiation detector is designed in order to detect the radiation, in particular the electromagnetic radiation. The radiation can for example pass from the radiation source to a region to be imaged and/or reach the radiation detector following an interaction with the region to be imaged. During the interaction with the region to be imaged the radiation is modified and thereby becomes the bearer of information relating to the region to be imaged. When the radiation interacts with the detector the information is captured in the form of acquisition data.

In particular in the case of a computed tomography device and in the case of a C-arm X-ray device the acquisition data can be projection data, the acquisition unit a projection data acquisition unit, the radiation source an X-ray source, and the radiation detector an X-ray detector. The X-ray detector can in particular be a quantum counting and/or energy resolved X-ray detector. In particular in the case of a magnetic resonance tomography device the acquisition data can be a magnetic resonance data set, the acquisition unit a magnetic resonance data acquisition unit, the radiation source a first radio-frequency antenna unit, the radiation detector the first radio-frequency antenna unit and/or a second radio-frequency antenna unit.

The component of the medical imaging apparatus can for example be a component of a patient positioning device of the medical imaging apparatus and/or a component of a gantry of the medical imaging apparatus. A component of the patient positioning device can for example be a drive which is designed in order to drive a movement of the transfer plate relative to the examination table. A component of the gantry can for example be a laser sight for positioning the patient and/or can be a radiation source. The component can in particular be designed in order to receive the control signal. In the case of a patient positioning device a control signal can for example effect a movement of the transfer plate. In the case of a radiation source a control signal can for example effect triggering of an irradiation.

The mobile control device can in particular comprise a housing. The housing can in particular be designed in such a manner that an interior space of the mobile control device is defined by the housing and is in particular limited in all directions.

The system can for example comprise a carrying unit which is designed for carrying the mobile control device on the body of a user. The carrying unit can for example comprise an element which is chosen from the group consisting of an armband, a belt, a lanyard and combinations thereof. The carrying unit can for example furthermore comprise a connection element which is designed in particular in order to connect in releasable fashion to the housing and/or to the docking module of the mobile control device. The mobile control device can then be designed in particular, if it comprises the carrying unit and/or is connected to the carrying unit, as a wearable.

The system can in particular comprise a further operating state in which the mobile control device and the control unit are not coupled or are not coupled in such a manner that an actuation of the operating element causes the control signal to be output to the component via the control unit. In particular, in the further operating state of the system it can be possible to couple the mobile control device and the control unit in such a manner that an actuation of the operating element causes the control signal to be output to the component via the control unit. The mobile control device and the control unit can in particular be coupled at least partially via hardware and/or at least partially via firmware and/or at least partially via software. The control unit can in particular be integrated into a control facility of the medical imaging apparatus and/or be arranged on the component of the medical imaging apparatus.

A context which relates to the medical imaging apparatus and/or a medical examination that can be carried out and/or is at least partially carried out via the medical imaging apparatus can in particular comprise an information item which relates to a state of the medical imaging apparatus and/or of the component of the medical imaging apparatus and/or a status of the examination, for example steps of the examination already performed and/or steps of the examination yet to be performed.

A context which relates to a patient positioning device can for example comprise an information item concerning whether or not the transfer plate has been inserted into the tunnel-shaped opening. A context which relates to the examination can for example comprise an information item specifying that during the next step of the predetermined workflow the transfer plate is to be inserted into the tunnel-shaped opening or that during the next step of the predetermined workflow the irradiation for the acquisition of acquisition data is to be triggered.

A pose of the mobile control device can in particular comprise an information item which relates to a position of the mobile control device and/or an orientation of the mobile control device. The pose of the mobile control device can for example be defined relative to an examination room in which the medical imaging apparatus is installed, and/or relative to a component of the medical imaging apparatus.

A position of the mobile control device can for example be understood as being a distance of the mobile control device from a reference location, for example on the gantry. In particular, it can be possible to configure the mobile control device and/or the control unit via the configuration unit in such a manner on the basis of the pose of the mobile control device that safety-related functions can no longer be initiated if the distance from the reference location exceeds a threshold value. The determination of the pose can for example take place on the basis of a triangulation, a field strength measurement or a transit time measurement for a data transfer medium in particular in the case of an IR and/or wireless based data transfer. In particular, in relation to the pose region boundary the determination of the pose can for example be based on the detection of an electronic resonant circuit and/or switching circuit via an antenna system.

Furthermore, the determination of the pose can for example take place on the basis of a determination of the docking unit to which the mobile control device is docked. This option can be expedient in particular in the situation when the medical imaging apparatus and/or the area surrounding the medical imaging apparatus comprises a plurality of docking units which are arranged spatially distributed and the positional information relating to the docking units is known to the system.

The releasable docking of the mobile control device to the docking unit can for example form a mechanical and/or form-fitting connection of the mobile control device to the docking unit, wherein the connection is in particular designed in such a manner that the forces and/or moments which act on the mobile control device when the operating elements are actuated are absorbed by the docking unit. In this manner the user is able to use one hand to operate the mobile control device which is docked to the docking unit. The releasable docking of the mobile control device to the docking unit can for example form an energy transfer connection and/or a data transfer connection between the mobile control device and the docking unit. For example, the mobile control device can be charged in cable-free fashion with electrical energy on the docking unit.

A solution according to at least one embodiment of the invention grants the user of a medical imaging apparatus a substantial degree of autonomy concerning how, when and from where he organizes his workflow. The user is for example thereby better able to adapt the workflow to the needs of the patient. For example, the user is able to a large extent to independently choose and flexibly adjust his position and orientation relative to the gantry and/or relative to the patient positioning device during the examination. The user is furthermore able to operate the mobile control device while walking or running, which offers a valuable time advantage in particular in emergency situations.

Operating elements which are designed as electromechanical switching elements additionally facilitate a greater degree of safety, in particular in relation to movements of the transfer plate and triggering of the irradiation, than for example operating elements which are embedded into a software application.

The operating element can be designed as an electromechanical switching element in particular if it comprises the electromechanical switching element. The electromechanical switching element can in particular be a switch, for example a pushbutton.

The actuation of the operating element can in particular comprise an actuation of the electromechanical switching element. The actuation of the electromechanical switching element can in particular reside in a mechanical switching operation during which for example an electrically conducting connection is established and/or disconnected and/or during which for example at least one mechanical and/or geometric parameter of an electronic switching circuit is modified.

In particular, when the system is in the operating state the mobile control device and the control unit can be coupled in such a manner that an actuation of the electromechanical switching element causes the control signal to be output to the component via the control unit.

The operating element can in particular comprise the electromechanical switching element and/or a touch-sensitive surface. The touch-sensitive surface of the operating element can in particular form a part of the touch-sensitive panel.

In particular, the component and/or a function can be assigned to the operating element on the basis of a touch gesture which is captured via the touch-sensitive surface of the operating element and/or via the touch-sensitive panel. In particular, an actuation of the operating element and/or an actuation of electromechanical switching element can effect the output of the control signal to the component via the control unit, where the control signal relates to the function which is assigned to the operating element.

In particular, a component of a system according to one of the embodiments which are disclosed in this description and/or in the claims, which is designed in order to perform a given step of a method according to one of the embodiments that are disclosed in this description and/or in the claims, can be implemented in the form of a hardware unit which is configured in order to perform the given step and/or which is configured in order to execute a computer-readable instruction in such a manner that the hardware unit can be configured via the computer-readable instruction in order to perform the given step. In particular, the system can comprise a storage area, for example in the form of a computer-readable medium in which computer-readable instructions are stored, for example in the form of a computer program.

The hardware in question can for example be a storage system, a FPGA system (field-programmable gate array), an ASIC system (application-specific integrated circuit), a microcontroller system, a processor system and combinations thereof. The processor system can for example comprise one microprocessor and/or a plurality of interacting microprocessors.

In the context of at least one embodiment of the invention, features which are described in relation to different embodiments of the invention and/or different categories of claims (method, use, device, system etc.) can be combined to produce further embodiments of the invention. For example, a claim which relates to a device can also be developed with features which are described or claimed in conjunction with a method. In this situation, functional features of a method can be performed by appropriately designed objective components. In addition to the embodiments of the invention expressly described in this application, diverse further embodiments of the invention are conceivable which the person skilled in the art can arrive at without departing from the scope of the invention insofar as it is predetermined by the claims.

The use of the indefinite article “a” or “an” does not mean that the feature in question cannot also be present several times. The use of the term “comprise” does not mean that the concepts linked by means of the term “comprise” cannot be identical. For example, the medical imaging apparatus comprises the medical imaging apparatus. The use of the term “unit” does not mean that the object to which the term “unit” relates cannot comprise a plurality of components which are spatially separated from one another.

The use of ordinal number words (first, second, third etc.) in the designation of features in the context of the present application serves primarily to distinguish more easily between the features designated using ordinal number words. The absence of a feature which is denoted by a combination of a given ordinal number word and a concept does not mean that a feature cannot be present which is denoted by a combination of a successor ordinal number word to the given ordinal number word and the concept.

The expression “on the basis of” in the context of the present application can in particular be understood in the sense of the expression “by use of”. In particular, a formulation according to which a first feature is produced (alternatively: determined, defined etc.) on the basis of a second feature does not mean that the first feature cannot be produced (alternatively: determined, defined etc.) on the basis of a third feature.

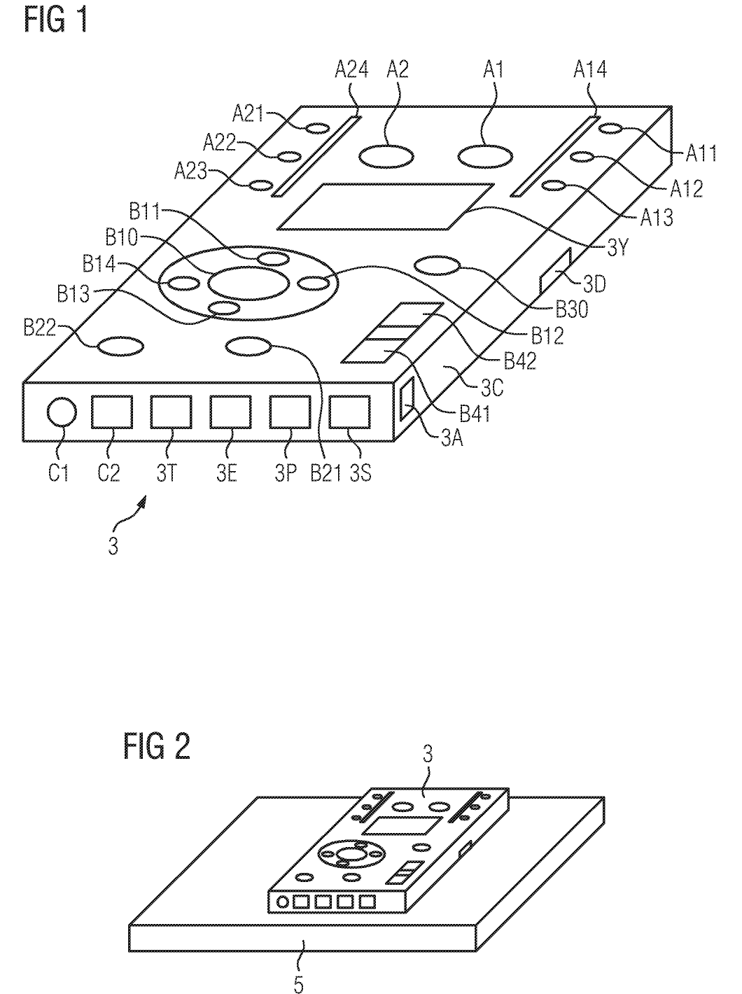

FIG. 1 shows a mobile control device 3 of a system 1 in accordance with a first embodiment of the invention.

The mobile control device 3 comprises a housing 3C. The housing 3C is designed in such a manner that an interior space of the mobile control device 3 is defined by the housing 3C and in particular is restricted in all directions. The housing 3C can for example be designed to be liquid-tight and/or liquid-resistant. The housing 3C can for example be designed as having antibacterial materials and/or be formed in such a manner as to avoid crevices which can only be disinfected with considerable effort.

Each of the operating elements B10, B11, B12, B13, B14, B21, B22, B30, B41, B42 is in each case designed as an electromechanical switching element on the housing 3C and/or can be actuated manually by a user U1. An electromechanical switching element can in particular comprise a pushbutton and/or a switch. Operation of the mobile control device 3 involves in particular the actuation of one or more of the operating elements.

The mobile control device 3 comprises one haptic button B10 which in context-sensitive fashion releases, activates or concludes the next respective step of a predetermined workflow. The predetermined workflow can include both hardware actions and also software actions. Examples of hardware actions are the loading and/or the unloading of the patient positioning device 10 and also the starting of a CT scan. Examples of software actions are the sending of the images from an examination in a PACS or the execution of the next step in each case in a sequence of consecutive work steps in a software application which for example is installed on the tablet computer 5 and/or on a smartphone and/or on the control facility 30.

An actuation of the operating element can in particular cause a control signal to be output to a component according to the function which is assigned to the operating element. The following functions are in particular assigned to the operating elements and/or display elements of the mobile control device 3:

B11: Raise the transfer plate 12, B13: Lower the transfer plate 12, B21: Load and/or Unload the patient positioning device 10, B22: Light switch, B30: Emergency Off switch, B41: Start the radiation exposure, B42: Stop the radiation exposure,

A11: Increase the sensitivity of the microphone A1, A12: Reduce the sensitivity of the microphone A1, A13: Marking which indicates to the user U1 that operating elements and display elements which relate to the microphone A1 are arranged in this area, A14: Display the sensitivity of the microphone A1,

A21: Increase the volume of the loudspeaker A2, A22: Reduce the volume of the loudspeaker A2, A23: Marking which indicates to the user U1 that operating elements and display elements which relate to the loudspeaker A2 are arranged in this area, A24: Display the volume of the loudspeaker A2.

The mobile control device 3 comprises a first transfer operating element B12 and a second transfer operating element B14. An actuation of the second transfer operating element B14 causes the transfer plate 12 to move in a horizontal direction which is opposite to the horizontal direction in which the transfer plate 12 is moved when the first transfer operating element B12 is actuated. In particular, the assignment of the horizontal directions to the transfer operating elements B12 and B14 can be effected on the basis of the pose of the mobile control device 3 relative to the transfer plate 12 and/or relative to the medical imaging apparatus 2.

For example, it can be possible to configure the mobile control device 3 and/or the control unit 35 via the configuration unit 7N in such a manner on the basis of the pose of the mobile control device 3 that for a first pose of the mobile control device 3 an actuation of the first transfer operating element B12 causes the transfer plate 12 to be inserted into the tunnel-shaped opening 9 and/or an actuation of the second transfer operating element B14 causes the transfer plate 12 to move away from the tunnel-shaped opening 9, and that for a second pose of the mobile control device 3 an actuation of the first transfer operating element B12 causes the transfer plate 12 to move away from the tunnel-shaped opening 9 and/or an actuation of the second transfer operating element B14 causes the transfer plate 12 to be inserted into the tunnel-shaped opening 9.

The first horizontal direction can for example point from the examination table 11 toward the tunnel-shaped opening 9. The movement of the transfer plate 12 in the first horizontal direction can in particular involve an insertion of the transfer plate 12 into the tunnel-shaped opening 9. The second horizontal direction can for example point from the tunnel-shaped opening 9 toward the examination table 11. The movement of the transfer plate 12 in the second horizontal direction can in particular involve a movement of the transfer plate 12 away from the tunnel-shaped opening 9.

In particular, the first pose in question can be a pose wherein the mobile control device 3 is situated on the same side as the patient positioning device 10 in relation to the gantry 20 and wherein the mobile control device 3 is oriented such that the first transfer operating element B12 is situated closer to the gantry 20 than the second transfer operating element B14.

In particular, the second pose in question can be a pose wherein the mobile control device 3 is situated on the same side as the patient positioning device 10 in relation to the gantry 20 and wherein the mobile control device 3 is oriented such that the second transfer operating element B14 is situated closer to the gantry 20 than the first transfer operating element B12.

The first pose of the mobile control device 3 can for example be present when the first transfer operating element B12 is situated to the right of the second transfer operating element B14 as viewed by the user U1, the user U1 is facing the patient positioning device 10 and the mobile control device 3 and the gantry 20 is situated to the right of the user U1. The second pose of the mobile control device 3 can for example be present when the first transfer operating element B12 is situated to the right of the second transfer operating element B14 as viewed by the user U1, the user U1 is facing the patient positioning device 10 and the mobile control device 3 and the gantry 20 is situated to the left of the user U1.

In other words, the assignment of the horizontal directions for the movement of the transfer plate 12 to the transfer operating elements B12 and B14 can depend on the side on which the user U1 is situated in relation to the transfer plate 12. The assignment can in particular be effected dynamically, for example by determining the pose of the mobile control device 3 regularly at short intervals and customizing the assignment on the basis of the pose. The assignment determined on the basis of the pose of the mobile control device 3 can in particular be displayed via the display unit 3Y. In this manner the user U1 is able to recognize which of the transfer operating elements B12 and B14 is to be actuated in order to move the transfer plate 12 in a predetermined direction.

Alternatively or additionally, each of the operating elements B10, B11, B12, B13, B14 can in each case be touch-sensitive and/or comprise a touch-sensitive surface. The operating elements B10, B11, B12, B13, B14 can thereby together form a touch-sensitive panel which is designed in order to display at least one software operating element and/or in order to capture at least one touch gesture in order to actuate at least one software operating element. The at least one software operating element can in particular be displayed via the display unit 3Y and/or via the touch-sensitive screen 5Y.

The touch-sensitive panel can in particular be composed of the touch-sensitive surfaces of the operating elements. The touch-sensitive panel can be contiguous or composed of a plurality of subpanels separated from one another.

By using the touch-sensitive panel it is possible in particular to navigate across a plurality of software elements, for example in order to control the component of the medical imaging apparatus 2 and/or to select data which is to be displayed to the user U1.

With the aid of the at least one touch gesture which is captured via the touch-sensitive panel it is possible in particular to scroll through a list containing data which for example relates to the patient 13 and/or an imaging protocol and/or an injection protocol. This can take place in particular by wiping the fingers across the touch-sensitive panel, for example upward and/or downward.

With the aid of the at least one touch gesture which is captured via the touch-sensitive panel it is possible in particular to confirm or cancel the next step in each case of a predetermined workflow which for example relates to the medical imaging examination and/or to control of the medical imaging apparatus 2 and/or is automatically selected by control software on the basis of the context. This can take place in particular by wiping the fingers across the touch-sensitive panel, for example to the right and/or to the left. In this manner, a software navigation forward and/or backward through the workflow can be implemented with the aid of the mobile control device 3.

A medical imaging examination can in particular be understood as being a medical examination which can be carried out and/or is at least partially carried out via the medical imaging apparatus.

The mobile control device 3 furthermore comprises the following components:

a terminal C1 for the cabled power supply,

a terminal C2 for cabled data transfer,

a data transfer module 3T,

an energy supply module 3E,

a data processing module 3P which comprises a memory module and a processor module, and

an alarm signal output unit 3A.

Instead of the separate terminals C1 and C2 it is possible for example to provide one terminal with which power supply and data transfer can take place over the same cable.

In particular, the data transfer module 3T can be situated entirely or partially in the interior space of the mobile control device 3. In particular, the energy supply module 3E can be situated entirely or partially in the interior space of the mobile control device 3. In particular, the data processing module 3P can be situated entirely or partially in the interior space of the mobile control device 3. In particular, one or more further components can be situated entirely or partially in the interior space of the mobile control device 3.

The mobile control device 3 comprises a dead man's device 3S which is designed in order to recognize and/or prevent an unintentional actuation of the operating element. An unintentional actuation of the operating element can for example result from the user U1 inadvertently sitting on the mobile control device 3.

The mobile control device 3 comprises a docking module 3D. The docking module 3D can in particular be designed universally in such a manner that the mobile control device 3 is able both to dock in releasable fashion via the docking module 3D to a docking unit of the medical imaging apparatus 2 and also to be connected to the tablet and/or to the carrying unit.

The mobile control device 3 comprises a first audio interface, having a microphone A1 and a loudspeaker A2, which is designed in order to receive acoustic signals from the user U1 and/or output acoustic signals to the user U1.

On the basis of the acoustic signals which are received by the user U1 via the first audio interface it is possible for example to determine voice commands for controlling the medical imaging apparatus 2. The voice commands can for example be determined by using voice recognition and processing software. The software can for example be installed on the mobile control device 3 and/or on the tablet computer 5 and/or on the control facility 30.

With the aid of the first audio interface the user U1 is also able to speak with the patient 13 and/or with other persons in the examination room from a greater distance and/or from another room, for example by using a second audio interface 40 which can be arranged on the gantry 20 and/or which can comprise a loudspeaker and/or a microphone.

The mobile control device 3 comprises a display unit 3Y which is designed in order to display to the user U1 data relating to the medical imaging apparatus 2 and/or the component of the medical imaging apparatus 2 and/or the examination and/or the patient 13. The display unit 3Y can for example be designed as a 7-segment display and/or as a screen. The mobile control device 3 can for example comprise a touch-sensitive screen which forms the display unit 3Y and an input unit. In particular, the display unit 3Y can be integrated into the housing 3C. By using the touch-sensitive screen it is possible for example to operate software for controlling the medical imaging apparatus 2 and/or the component of the medical imaging apparatus 2.

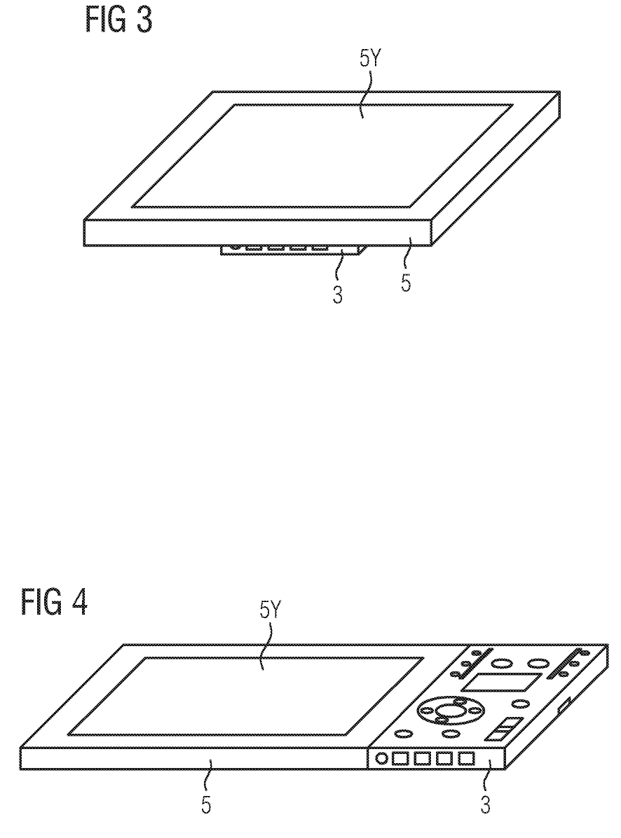

FIG. 2 shows a first view of a mobile control device 3 of a system 1 in accordance with a second embodiment of the invention. FIG. 3 shows a second view of a mobile control device 3 of a system 1 in accordance with the second embodiment of the invention.

The mobile control device 3 can be used together with a tablet computer 5 and/or a smartphone on which a software application is installed which is designed in order to control the medical imaging apparatus 2 and/or the component of the medical imaging apparatus 2.

The tablet computer 5 comprises the touch-sensitive screen 5Y. With the aid of the tablet computer it is possible in particular to perform one or more of the following steps of the workflow of a medical imaging examination:

registration of the patient 13,

adaptation of examination parameters, for example of the imaging protocol and/or of the injection protocol,

image processing and/or finalization of medical image data sets,

use of further radiology software, in particular in conjunction with PACS, RIS, HIS,

communication with other users, in particular with colleagues in other clinical departments, for example radiology, reception, accident and emergency, or similar.

The mobile control device 3 can for example be docked magnetically to the tablet computer 5 and/or to a smartphone. In this manner the tablet computer 5 and the mobile control device 3 can be carried comfortably in only one hand. In this manner the smartphone and the mobile control device 3 can be carried comfortably in only one hand.

FIG. 4 shows a mobile control device 3 of a system 1 in accordance with a third embodiment of the invention.

The mobile control device 3 can for example comprise the tablet computer 5 or be fixedly connected to a tablet computer 5 to form a single control module. The control module enables the user U1 to interact with the medical imaging apparatus 2 both via electromechanical switching elements, which can be advantageous in particular for safety-related control commands, and also via the software application installed on the tablet computer 5, which can be advantageous in particular in the case of more complex applications which are not safety-critical.

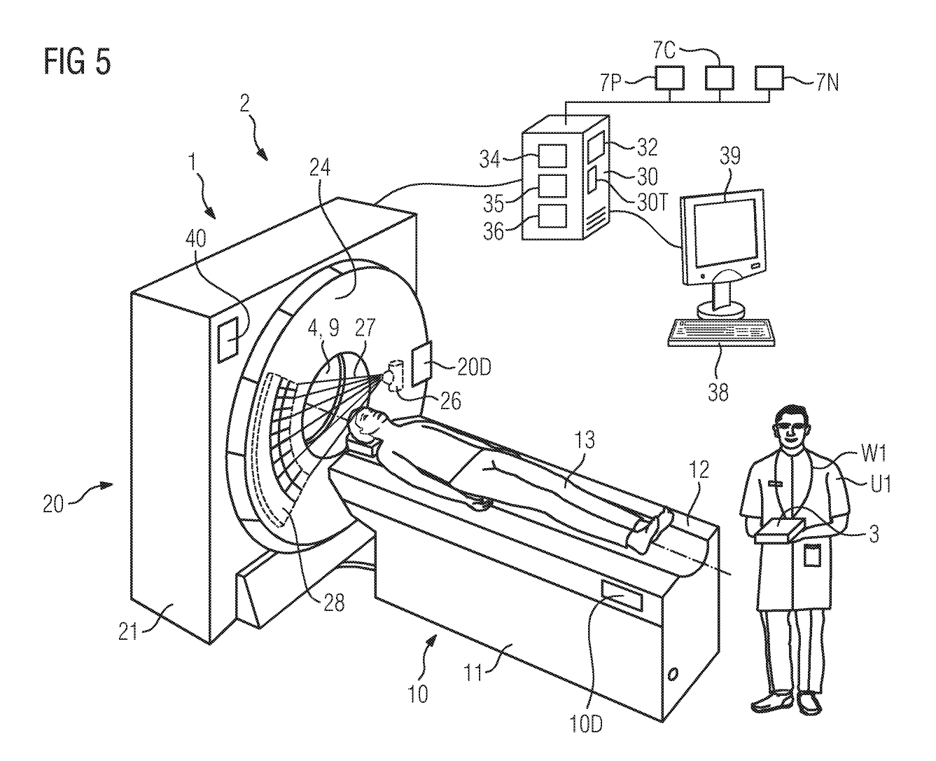

FIG. 5 shows a system 1 in accordance with a fourth embodiment of the invention, comprising the medical imaging apparatus 2.

Without limiting the generality of embodiments of the invention, a computed tomography device is shown by way of example for the medical imaging apparatus 2. The medical imaging apparatus 2 comprises the gantry 20, the tunnel-shaped opening 9, the patient positioning device 10 and the control facility 30.

The gantry 20 comprises the stationary supporting frame 21 and the rotor 24. The rotor 24 is arranged in rotatable fashion about an axis of rotation relative to the stationary supporting frame 21 via a rotational bearing arrangement on a tilting frame.

The patient 13 can be introduced into the tunnel-shaped opening 9. The acquisition region 4 is situated in the tunnel-shaped opening 9. A region to be imaged of the patient 13 can be positioned in such a manner in the acquisition region 4 that the radiation 27 can pass from the radiation source 26 to the region to be imaged and following an interaction with the region to be imaged can pass to the radiation detector 28.

The patient positioning device 10 comprises the examination table 11 and the transfer plate 12 for positioning the patient 13. The transfer plate 12 is arranged in movable fashion on the examination table 11 in such a manner relative to the examination table 11 that the transfer plate 12 can be introduced into the acquisition region 4 in a longitudinal direction of the transfer plate 12.

The medical imaging apparatus 2 is designed in order to acquire acquisition data on the basis of electromagnetic radiation 27. The medical imaging apparatus 2 comprises an acquisition unit. The acquisition unit is a projection data acquisition unit having the radiation source 26, for example an X-ray source, and the detector 28, for example an X-ray detector, in particular an energy resolved X-ray detector. The radiation source 26 is arranged on the rotor 24 and is designed in order to emit radiation 27, for example X-ray radiation, with radiation quanta 27. The detector 28 is arranged on the rotor 24 and is designed in order to detect the radiation quanta 27. The radiation quanta 27 can pass from the radiation source 26 to the region to be imaged of the patient 13 and following an interaction with the region to be imaged reach the detector 28. In this manner, acquisition data from the region to be imaged can be captured in the form of projection data via the acquisition unit.

The control facility 30 is designed in order to receive the acquisition data acquired by the acquisition unit. The control facility 30 is designed in order to control the medical imaging apparatus 2. In accordance with the fourth embodiment of the invention the control unit 35 is integrated into the control facility 30.

The control facility 30 is formed by a data processing system which comprises a computer, and comprises the computer-readable medium 32 and the processor system 36.

The control facility 30 comprises the image reconstruction facility 34. A medical image data set can be reconstructed via the image reconstruction facility 34 on the basis of the acquisition data.

The medical imaging apparatus 2 comprises an input facility 38 and an output facility 39, each of which is connected to the control facility 30. The input facility 38 is designed in order to enter control information, for example image reconstruction parameters, examination parameters or the like. The output facility 39 is designed in particular in order to output control information, images and/or acoustic signals.

The system 1 comprises a data transfer unit which is designed in order to transfer data between the control unit 35 and the mobile control device 3. The data can include for example acquisition parameters, in particular imaging protocol parameters, injection protocol parameters, patient parameters, in particular from the electronic medical record of the patient 13, and/or measurement data from measurements supporting the medical imaging examination, in particular from an ECG measurement. The data transfer unit comprises the data transfer module 3T of the mobile control device 3 and the data transfer module 30T of the control facility 30, wherein each of the data transfer modules 3T and 30T is designed in each case in order to send and/or receive the data. The data can for example be transferred in cable-free and/or cabled fashion. The data can in particular be transferred on the basis of a communication protocol which is bidirectional and/or first-fail-safe.

In this manner, an embodiment of the invention enables cable-free safe remote control of the medical imaging apparatus 2 and/or of a component of the medical imaging apparatus 2. Use of the bidirectional communication protocol means for example that in addition to the data which relates to control of the component and which is transferred from the mobile control device 3 to the control unit 35 further data which relates for example to the state of one or more components of the medical imaging apparatus 2 can be transferred to the mobile control device 3. This means for example that released movements of the transfer plate and/or a state of a laser sight for patient positioning can be displayed to the user U1 via the display unit of the mobile control device 3.

The system 1 furthermore comprises the context determination unit 7C, the pose determination unit 7P and the configuration unit 7N. In accordance with the fourth embodiment of the invention the system 1 comprises a carrying unit W1 having a lanyard. The lanyard allows the user U1 to carry the mobile control device 3 on his body. The medical imaging apparatus 2 comprises a docking unit 20D on the gantry 20 and a docking unit 10D on the patient positioning device 10. Each of the docking units 20D and 10D is designed for releasable docking of the mobile control device 3.

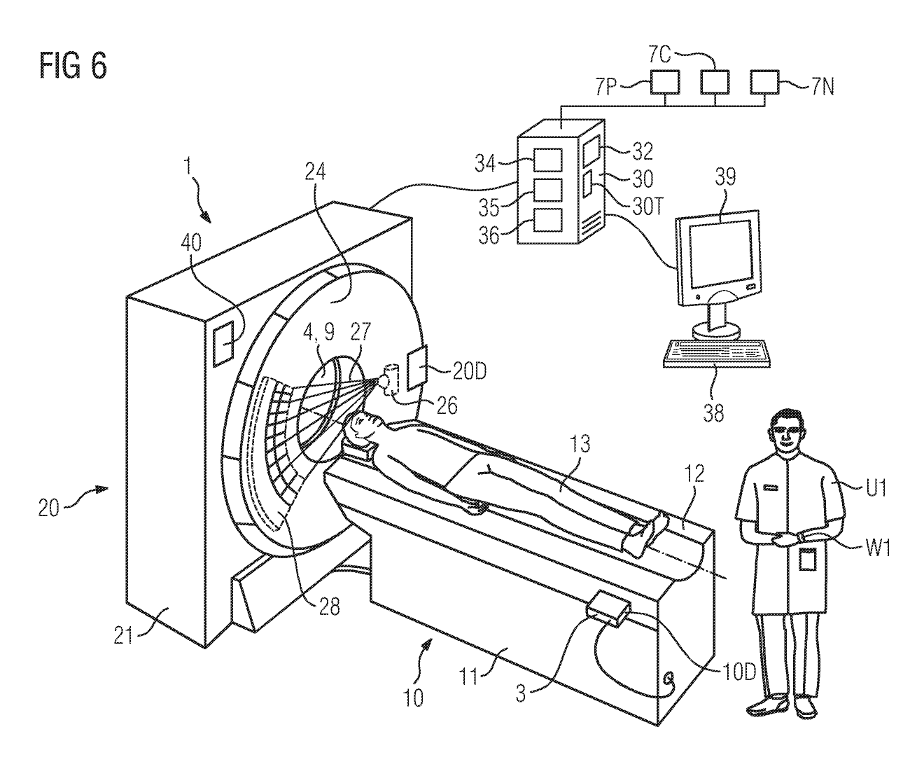

FIG. 6 shows a system 1 in accordance with a fifth embodiment of the invention, wherein the mobile control device 3 is arranged on the docking unit 10D. In accordance with the fifth embodiment of the invention the mobile control device 3 is connected via a cable to the medical imaging apparatus 2, wherein a power supply and/or data transfer facility can be provided by way of the cable.

In accordance with the fifth embodiment of the invention the system 1 comprises a carrying unit W1 having an armband. The armband allows the user U1 to carry the mobile control device 3 on his body, in particular on the wrist.

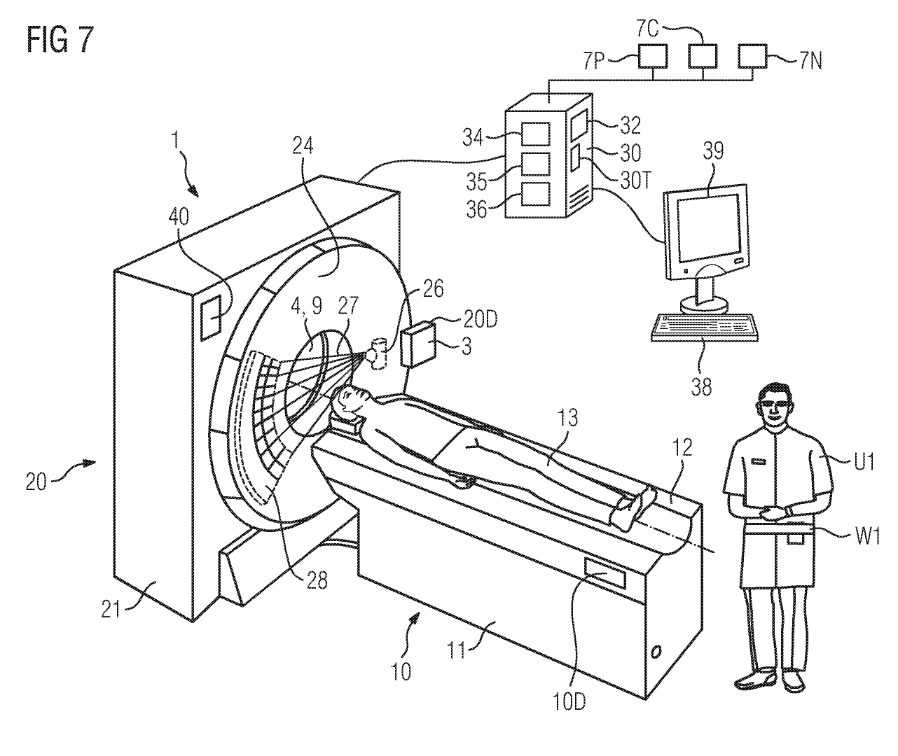

FIG. 7 shows a system 1 in accordance with a sixth embodiment of the invention, wherein the mobile control device 3 is arranged on the docking unit 20D. In accordance with a sixth embodiment of the invention the system 1 comprises a carrying unit W1 having a belt. The belt allows the user U1 to carry the mobile control device 3 on his body.

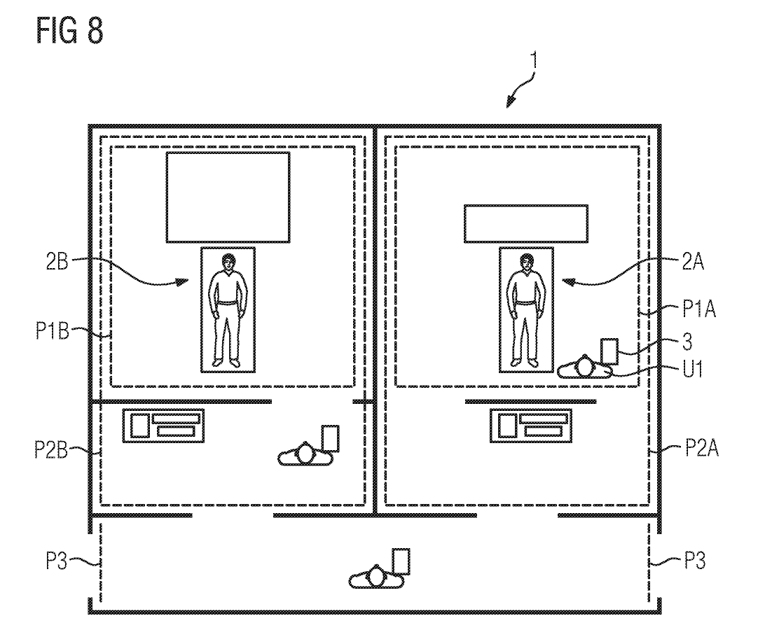

FIG. 8 shows a system 1 in accordance with a seventh embodiment of the invention, wherein the system 1 comprises two medical imaging apparatuses.

The mobile control device 3 can for example be used in a system 1 having a plurality of medical imaging apparatuses. The medical imaging apparatuses can in particular include different imaging modalities, for example CT devices and MR devices. In particular, the mobile control device 3 can be designed in order to recognize in which room and/or in the vicinity of which of the plurality of medical imaging apparatuses it is situated, and/or to connect to the respective medical imaging apparatus, in particular automatically, for example using “Plug & Play”.

In particular, it is possible to use the display unit 3Y of the mobile control device 3 to display to which of the plurality of medical imaging apparatuses and/or to which of the different imaging modalities the mobile control device 3 is connected.

The system 1 can in particular comprise a plurality of medical imaging apparatuses and a selection unit which is designed in order to select one medical imaging apparatus 2 from the plurality of medical imaging apparatuses on the basis of the context and/or on the basis of the pose of the mobile control module. The selection unit can for example be integrated into the mobile control device 3 and/or into a data processing system, which can be connected in particular to the mobile control device 3 and/or to the control units of the medical imaging apparatuses via a data transfer connection.

In particular, each medical imaging apparatus of the plurality of medical imaging apparatuses can comprise a respective control unit which is designed in order to output a control signal to a component of the respective medical imaging apparatus. The system 1 can in particular be designed in order to couple the mobile control device 3 and the control unit of the selected medical imaging apparatus in such a manner that an actuation of the operating element of the mobile control device 3 causes the control signal to be output to the component via the control unit.

The pose determination unit 7P is designed in order to determine whether the pose of the mobile control device 3 is situated in a first pose region and/or in a second pose region and/or whether the pose of the mobile control device 3 crosses the pose region boundary P3. For the medical imaging apparatus 2A, the first pose region P1A and the second pose region P2A are each illustrated by dashed lines. For the medical imaging apparatus 2B, the first pose region P1B and the second pose region P2B are each illustrated by dashed lines.

In accordance with the seventh embodiment of the invention provision is made that the first pose region includes the examination room in which the medical imaging apparatus is installed and that the second pose region includes the examination room and the control room which is at least partially separated from the examination room via a dividing wall.

In accordance with the seventh embodiment of the invention, an actuation of the corresponding operating element, for example the first transfer operating element or the second transfer operating element, only causes a control signal for moving the transfer plate to be output to the patient positioning device if the pose of the mobile control device 3 is situated in the corresponding first pose region.

It is possible to choose a plurality of first pose regions, each of which is assigned to a component and/or an operating element. For example, a different first pose region can be chosen which includes only the control room, in which case actuation of the corresponding operating element only causes a control signal for triggering the irradiation to be output to the radiation source when the pose of the mobile control device 3 is situated in the control room.

In accordance with the seventh embodiment of the invention the mobile control device 3 and the control unit are only coupled when the pose of the mobile control device 3 is situated in the corresponding second pose region.

In accordance with the seventh embodiment of the invention, if the pose of the mobile control device 3 crosses the pose region boundary P3 an alarm signal is output via the alarm signal output unit 3A, for example in the form of an alarm tone and/or a vibration alarm. In order to turn off the alarm signal, it may be necessary for example to cross the pose region boundary P3 again and/or further measures may be required. In this manner it is possible to avoid the mobile control device 3 being inadvertently taken away by a user U1.

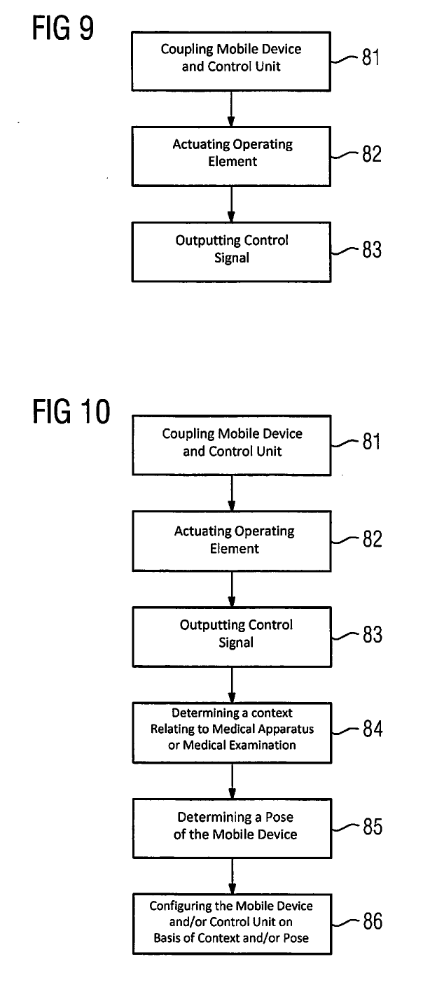

FIG. 9 shows a flowchart for a method for outputting a control signal to a component of a medical imaging apparatus 2 in accordance with an eighth embodiment of the invention, wherein the method comprises:

coupling 81 a mobile control device 3, which comprises an operating element that is designed as an electromechanical switching element, and a control unit 35, which is designed in order to output a control signal to the component, in such a manner that an actuation of the operating element causes the control signal to be output to the component via the control unit 35,

actuating 82 the operating element, and

outputting 83 the control signal.

FIG. 10 shows a flowchart for a method for outputting a control signal to a component of a medical imaging apparatus 2 in accordance with a ninth embodiment of the invention, wherein the method furthermore involves the following steps:

determining 84 a context which relates to the medical imaging apparatus 2 and/or a medical examination that can be performed via the medical imaging apparatus 2, and/or determining 85 a pose of the mobile control device 3,

configuring 86 the mobile control device 3 and/or the control unit 35 on the basis of the context and/or on the basis of the pose of the mobile control device 3.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A system, comprising
a component of a medical imaging apparatus;
a mobile control device including an operating element designed as an electromechanical switching element; and
a control unit designed to output a control signal to the component, wherein, when the system is in an operating state, the mobile control device and the control unit are coupled such that an actuation of the operating element is designed to cause the control signal to be output to the component via the control unit.

2. The system of claim 1, wherein the mobile control device is designed such that at least one of:
the mobile control device is carriable in one hand or on the body by a user and
the mobile control device is operatable by a user while the mobile control device is being carried in at least one of one hand and on the body by the user.

3. The system of claim 1, wherein the mobile control device comprises a first audio interface, designed to at least one of receive acoustic signals from the user and output acoustic signals to the user.

4. The system of claim 1, wherein the mobile control device comprises a plurality of touch-sensitive operating elements forming a touch-sensitive panel, designed to at least one of display at least one software operating element and capture at least one touch gesture for actuating at least one software operating element.

5. The system of claim 1, wherein the mobile control device comprises a display unit to display to the user, of data relating to at least one the medical imaging apparatus and the component of the medical imaging apparatus.

6. The system of claim 1, further comprising at least one of
at least one context determination unit to determine at least one of a context relating to the medical imaging apparatus and a medical examination capable of being carried out via the medical imaging apparatus; and
a pose determination unit to determine a pose of the mobile control device.

7. The system of claim 6, wherein the pose determination unit is designed to determine whether the pose of the mobile control device crosses a pose region boundary, the system further comprising an alarm signal output unit, designed to output an alarm signal such that the alarm signal is output when the pose of the mobile control device crosses a pose region boundary.

8. The system of claim 6, further comprising
a configuration unit designed to configure at least one of the mobile control device and the control unit on the basis of at least one of the context and the pose of the mobile control device.

9. The system of claim 8,
wherein the component of the medical imaging apparatus comprises an examination table and a transfer plate, arranged on the examination table and movable relative to the examination table,
wherein the mobile control device comprises a first transfer operating element, and
wherein at least one of the mobile control device and the control unit is configurable via the configuration unit, on the basis of the pose of the mobile control device, such that for at least a first pose of the mobile control device, an actuation of the first transfer operating element is designed to cause the transfer plate to move in a first horizontal direction and such that for at least a second pose of the mobile control device, an actuation of the first transfer operating element is designed to cause the transfer plate to move in a second horizontal direction.

10. The system of claim 8,
wherein the pose determination unit is designed to determine whether the pose of the mobile control device is situated in a first pose region, and
wherein at least one of the mobile control device and the control unit is configurable via the configuration unit such that, on the basis of the pose of the mobile control device, an actuation of the operating element is designed to cause the control signal to be output to the component via the control unit depending on whether or not the pose of the mobile control device is situated in the first pose region.

11. The system of claim 8,
wherein the pose determination unit is designed to determine whether the pose of the mobile control device is situated in a second pose region, and
wherein at least one of the mobile control device and the control unit is configurable via the configuration unit such that, on the basis of the pose of the mobile control device, the mobile control device and the control unit are coupled depending on whether or not the pose of the mobile control device is situated in the second pose region.

12. The system of claim 1, further comprising
at least one of a tablet computer and a smartphone including a software application designed to control at least one of the medical imaging apparatus and a component of the medical imaging apparatus, wherein at least one of
the mobile control device comprises the at least one of the tablet computer and the smartphone, and
the mobile control device is conectable to the at least one of the tablet computer and the smartphone via at least one of a mechanical connection and a data transfer connection.

13. The system of claim 1, further comprising
the medical imaging apparatus, wherein the medical imaging apparatus comprises a docking unit designed to dock the mobile control device in releasable fashion.

14. The system of claim 12, wherein the mechanical connection includes a magnetic holder.

15. A method for outputting a control signal to a component of a medical imaging apparatus, the method comprising:
coupling a mobile control device, including an operating element, designed as an electromechanical switching element, and a control unit, designed to output a control signal to the component, in such a manner that an actuation of the operating element is designed to cause the control signal to be output to the component via the control unit;
actuating the operating element; and
outputting the control signal.

16. The method of claim 15, further comprising:
at least one of
determining a context relating to at least one of the medical imaging apparatus and a medical examination designed to be carried out via the medical imaging apparatus, and
determining of a pose of the mobile control device; and
configuring at least one of the mobile control device and the control unit on the basis of at least one of the context and the pose of the mobile control device.

17. A method, comprising:
using the system of claim 1 to output a control signal to a component of a medical imaging apparatus, wherein the output of the control signal to the component via the control unit is effected by actuating the operating element.

18. A non-transitory computer readable medium including program code for carrying out the method of claim 15 when the program code is run on a computer.

19. The system of claim 2, wherein the mobile control device comprises a first audio interface, designed to at least one of receive acoustic signals from the user and output acoustic signals to the user.

20. The system of claim 2, wherein the mobile control device comprises a plurality of touch-sensitive operating elements which form a touch-sensitive panel designed to at least one of display at least one software operating element and capture at least one touch gesture for actuating at least one software operating element.

21. The system of claim 3, wherein the mobile control device comprises a plurality of touch-sensitive operating elements which form a touch-sensitive panel designed to at least one of display at least one software operating element and capture at least one touch gesture for actuating at least one software operating element.

22. The system of claim 2, further comprising at least one of
at least one context determination unit to determine at least one of a context relating to the medical imaging apparatus and a medical examination capable of being carried out via the medical imaging apparatus; and
a pose determination unit to determine a pose of the mobile control device.

23. The system of claim 22, wherein the pose determination unit is designed to determine whether the pose of the mobile control device crosses a pose region boundary, the system further comprising an alarm signal output unit, designed to output an alarm signal such that the alarm signal is output when the pose of the mobile control device crosses a pose region boundary.

24. The system of claim 22, further comprising
a configuration unit designed to configure at least one of the mobile control device and the control unit on the basis of at least one of the context and the pose of the mobile control device.

25. The system of claim 7, further comprising
a configuration unit designed to configure at least one of the mobile control device and the control unit on the basis of at least one of the context and the pose of the mobile control device.

26. The system of claim 10,
wherein the pose determination unit is designed to determine whether the pose of the mobile control device is situated in a second pose region, and
wherein at least one of the mobile control device and the control unit is configurable via the configuration unit such that, on the basis of the pose of the mobile control device, the mobile control device and the control unit are coupled depending on whether or not the pose of the mobile control device is situated in the second pose region.

27. The system of claim 2, further comprising
at least one of a tablet computer and a smartphone including a software application designed to control at least one of the medical imaging apparatus and a component of the medical imaging apparatus, wherein at least one of
the mobile control device comprises the at least one of the tablet computer and the smartphone, and
the mobile control device is conectable to the at least one of the tablet computer and the smartphone via at least one of a mechanical connection and a data transfer connection.

28. The system of claim 13, wherein the docking unit includes a magnetic holder.

29. The method of claim 16,
wherein the determining of the pose includes determining whether the pose of the mobile control device is situated in a first pose region, and
wherein at least one of the mobile control device and the control unit is configurable such that, on the basis of the pose of the mobile control device, an actuation of the operating element is designed to cause the control signal to be output to the component depending on whether or not the pose of the mobile control device is situated in the first pose region.

30. The method of claim 16,
wherein the determining of the pose includes determining whether the pose of the mobile control device is situated in a second pose region, and
wherein at least one of the mobile control device and the control unit is configurable such that, on the basis of the pose of the mobile control device, the mobile control device and the control unit are coupled depending on whether or not the pose of the mobile control device is situated in the second pose region.

31. The method of claim 29,
wherein the determining of the pose includes determining whether the pose of the mobile control device is situated in a second pose region, and
wherein at least one of the mobile control device and the control unit is configurable such that, on the basis of the pose of the mobile control device, the mobile control device and the control unit are coupled depending on whether or not the pose of the mobile control device is situated in the second pose region.
